(19) United States
(12) Patent Application Publication
(10) Pub. No.: US 2009/0012105 A1
(43) Pub. Date: Jan. 8, 2009

(54) Combination of Mtor Inhibitor and Antipolate Compound

(75) Inventors: Heidi Lane, Biel-Benken (CH); Terence O'Reilly, Basel (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, BASEL (CH)

(21) Appl. No.: 12/087,385

(22) PCT Filed: Jan. 12, 2008
(86) PCT No.: PCT/EP2007/000273

§ 371 Date: Jul. 3, 2008

(30) Foreign Application Priority Data
Jan. 12, 2006 (GB) 0600618.3
May 18, 2006 (GB) 0609914.7
Jul. 14, 2006 (EP) 06117276.3

(51) Int. Cl. A61K 31/519; A61K 31/436

(52) U.S. Cl. 514/265.1; 514/291

(57) ABSTRACT

Use of a combination of an mTOR inhibitor and an antifolate compound.

The present invention relates to a cancer combination treatment, e.g. to a combination of pharmaceutically active organic compounds, e.g. a combination of antifolate compounds and mTOR inhibitors.

An mTOR inhibitor as used herein is a compound which targets intracellular mTOR (“mammalian Target of rapamycin”). mTOR is a family member of phosphatidylinositol 3-kinase (P13-kinase) related kinase. The compound rapamycin and other mTOR inhibitors inhibit mTOR activity via a complex with its intracellular receptor FKBP12 (FK506-binding protein 12). mTOR modulates translation of specific mRNAs via the regulation of the phosphorylation state of several different translation proteins, mainly 4E-PB1, P70S6K (p70S6 kinase 1) and eEF2.

An mTOR inhibitor as used herein includes rapamycin and rapamycin derivatives.

Rapamycin is a known macrolide antibiotic produced by Streptomyces hygroscopicus of formula

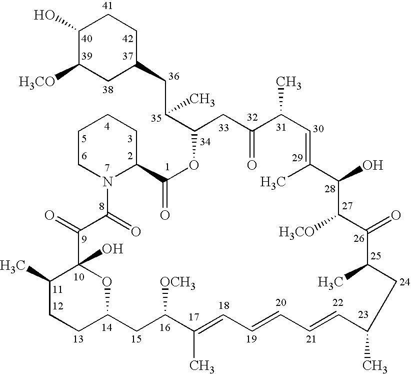

Rapamycin derivatives include for example rapamycin substituted in position 40 and/or 16 and/or 32.

Examples of rapamycin derivatives include 40-O-alkyl-rapamycin derivatives, e.g. 40-O-hydroxyalkyl-rapamycin derivatives, for example 40-O-(2-hydroxy)-ethyl-rapamycin (everolimus), herein also designated as “Compound A”,

rapamycin derivatives which are substituted in 40 position by heterocyclyl, e.g. 40-epi-(tetrazolyl)-rapamycin (also known as ABT578),
32-deoxo-rapamycin derivatives and 32-hydroxy-rapamycin derivatives, such as 32-deoxorapamycin,
16-O-substituted rapamycin derivatives such as 16-pent-2-ynyloxy-32-deoxorapamycin, 16-pent-2-ynyloxy-32(S or R)-dihydro-rapamycin, or 16-pent-2-ynyloxy-32(S or R)-dihydro-40-O-(2-hydroxyethyl)-rapamycin,
rapamycin derivatives which are acylated at the oxygen in position 40, e.g. 40-[3-hydroxy-2-(hydroxy-methyl)-2-methylpropanoate]-rapamycin (also known as CCI779 or temsirolimus),
rapamycin derivatives (also sometimes designated as rapalogs) as disclosed in WO9802441 or WO0114387, e.g. including AP23573, such as 40-O-dimethylphosphinyl-rapamycin,
compounds disclosed under the name biolimus (biolimus A9), including 40-O-(2-ethoxy)ethyl-rapamycin, and compounds disclosed under the name TAFA-93, AP23464, AP23675 or AP23841; or
rapamycin derivatives as e.g. disclosed in WO2004101583, WO9205179, WO9402136, WO9402385 and WO9613273.

Preferred mTOR inhibitors include

rapamycin, and/or
40-O-(2-hydroxyethyl)-rapamycin, and/or
32-deoxorapamycin, and/or
16-pent-2-ynyloxy-32-deoxorapamycin, and/or
16-pent-2-ynyloxy-32 (S or R)-dihydro-rapamycin, and/or
16-pent-2-ynyloxy-32 (S or R)-dihydro-40-O-(2-hydroxyethyl)-rapamycin, and/or
40-[3-hydroxy-2-(hydroxy-methyl)-2-methylpropanoate]-rapamycin (also known as CCI779) and/or
40-epi-(tetrazolyl)-rapamycin (also known as ABT578), and/or
the so-called rapalogs, e.g. as disclosed in WO9802441, WO0114387 and WO0364383, AP23573, AP23464, AP23675 or AP23841, e.g. AP23573, and/or
compounds disclosed under the name TAFA-93, and/or
compounds disclosed under the name biolimus.

More preferably an mTOR inhibitor is selected from the group consisting of

rapamycin, and/or
40-O-(2-hydroxyethyl)-rapamycin, and/or
32-deoxorapamycin, and/or
16-pent-2-ynyloxy-32-deoxorapamycin, and/or
16-pent-2-ynyloxy-32 (S or R)-dihydro-rapamycin, and/or
16-pent-2-ynyloxy-32 (S or R)-dihydro-40-O-(2-hydroxyethyl)-rapamycin, and/or
40-[3-hydroxy-2-(hydroxy-methyl)-2-methylpropanoate]-rapamycin (also known as CCI779) and/or
40-epi-(tetrazolyl)-rapamycin (also known as ABT578), and/or

### AP23573,

preferably 40-O-(2-hydroxyethyl)-rapamycin.

mTOR inhibitors, on the basis of observed activity, have been found to be useful as pharmaceuticals, e.g. as immunosuppressants, for example for the treatment of conditions following transplantation; as anti-inflammatory compounds, e.g. for the treatment of IBD, RA; as anti-allergic compounds, e.g. for the treatment of psoriasis, and have additionally potent antiproliferative properties which make them useful for cancer chemotherapy, such as for the treatment of solid tumors, especially of advanced solid tumors.

Rapamycin and other rapamycin derivatives may be administered as appropriate, e.g. in dosages which are known for rapamycin or rapamycin derivatives, e.g. everolimus may be administered, e.g. orally, in dosages from 0.1 mg up to 25 mg, e.g. 1 mg up to 15 mg, such as 0.1 mg to 10 mg. e.g. 0.1 mg, 0.25 mg, 0.5 mg, 0.75 mg, 1 mg, 2.5 mg, 5 mg, or 10 mg, preferably 1 mg to 10 mg, e.g. in the form of (dispersible) tablets; e.g. a weekly dosage may include up to 70 mg, e.g. 30 mg to 70 mg, such as 30 mg to 50 mg; depending on the disease being treated. Other rapamycin derivatives may be administered in similar dosage ranges.

Antifolate compounds are known and have an inhibitory effect on one or more enzymes which utilize folic acid, and in particular metabolic derivatives of folic acid, as a substrate. An antifolate compound as used herein e.g. include compounds as described in U.S. Pat. No. 5,344,932, for example including a compound of formula

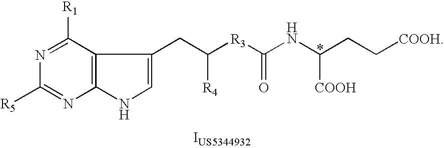

wherein

### R₁ is —OH or —NH₂;

R₃ is 1,4-phenylene or 1,3-phenylene unsubstituted or substituted with chloro, fluoro, methyl, methoxy, or trifluoromethyl; thienediyl or furanediyl unsubstituted or substituted with chloro, fluoro, methyl, methoxy, or trifluoromethyl; cyclohexanediyl; or alkanediyl;
R₄ is hydrogen, methyl, or hydroxymethyl; and
R₅ is hydrogen, alkyl of 1 to 6 carbon atoms, or amino;
e.g. the configuration of the substituent at the carbon atom designated * is S;
e.g. in free form or in the form of a pharmaceutically acceptable salt; optionally in the form of a solvate;
preferably the compound of formula

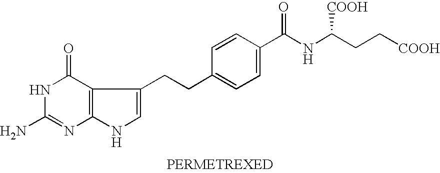

e.g. in the form of a sodium salt, such as a disodium salt, e.g. in the form of a solvate, such as a hydrate, e.g. a heptahydrate.

A compound of formula PERMETREXED is known under the name permetrexed and is approved for injection in the form of a disodium salt heptahydrate under the trade name Alimta®.

Antifolate compounds of formula I appear to be particularly active as inhibitors of thymidylate synthetase, which catalyses the methylation of deoxyuridylic acid to deoxythymidylic acid utilizing N⁵,N¹⁰-methylidenetetrahydrofolate as a coenzyme. The compounds thus can be used to inhibit the growth of those neoplasms which otherwise depend upon the inhibited enzyme.

The compounds of U.S. Pat. No. 5,344,932 are described to inhibit the growth of neoplasms including choriocarcinoma, leukemia, adenocarcinoma of the female breast, epidermid cancers of the head and neck, squamous or small-cell lung cancer, and various lymphosarcomas. The compounds of U.S. Pat. No. 5,344,932 are also described to be useful for the treatment of mycosis fungoides, a type of skin cancer; and psoriasis.

Specifically Alimta® is recommended for the treatment of (malignant) mesothelioma, which is a cancer of mesothelial cells, e.g. which cancer can developed in the pleura, (pleural mesothelioma), abdomen, e.g. mesothelioma of the tissues lining the abdominal cavity (peritoneal mesothelioma), and the lining of the lungs, or in the lining of the reproductive organs (begnin mesothelioma), recurrent non-small cell lung cancer (NSCLC), gestational trophoblastic tumor.

The recommended dose of ALIMTA® is 500 mg/m² administered as an intravenous infusion over 10 minutes on Day 1 of each 21-day cycle, e.g. for the treatment non small lung cancer, e.g. or in combination with cisplatin for the treatment of mesothelioma, e.g. malignant pleural mesothelioma (the recommended dose of cisplatin is 75 mg/m² infused over 2 hours beginning approximately 30 minutes after the end of ALIMTA administration).

A limitation to the development of antifolate compounds is that the cytotoxic activity and subsequent effectiveness of antifolates may be associated with substantial toxicity for some patients. Additionally antifolates as a class are associated with sporadic severe mylosuppression with gastrointestinal toxicity which, though infrequent, carries a high risk of mortality. The inability to control these toxicities led to the abandonment of clinical development of some antifolates and has complicated the clinical development of others.

It was now found that a combination of an mTOR inhibitor and an antifolate, e.g. a compound of formula I U.S. Pat. No. 5,344,932, shows surprising results in the treatment of conditions for which mTOR inhibitors and/or a compound of formula I U.S. Pat. No. 5,344,932 are useful, e.g. for the treatment of inflammatory or (auto)immune disorders, such as e.g. psoriasis and for the treatment of cancer, e.g. for the treatment of tumors.

Surprisingly certain toxic effects caused by antifolates of formula I U.S. Pat. No. 5,344,932, may be reduced by the presence of an mTOR-inhibitor, without adversely affecting therapeutic efficacy. Surprisingly a combined treatment with a compound of formula I U.S. Pat. No. 5,344,932 and an mTOR inhibitor synergistically may reduce the amount of each single drug, if administered in combination, compared with single treatment for achieving similar effects. Surprisingly the combination of an mTOR inhibitor and a compound of formula I U.S. Pat. No. 5,344,932 may reduce the toxic events associated with administration of antifolate drugs of formula I U.S. Pat. No. 5,344,932, e.g. synergistically.

In different aspects the present invention provides
1.1 A combination comprising an mTOR inhibitor and an antifolate compound.

A combination provided by the present invention includes a pharmaceutical combination. In another aspect the present invention provides
1.2 A pharmaceutical combination comprising an mTOR inhibitor and an antifolate compound.

A (pharmaceutical) combination provided by the present invention includes
fixed combinations, in which two or more pharmaceutically active agents, e.g. an mTOR inhibitor and an antifolate compound are in the same formulation,
kits, in which two or more pharmaceutically active agents, e.g. an mTOR inhibitor and a an antifolate compound in separate formulations are sold in the same package, e.g. with instruction for co-administration; and
free combinations in which two or more pharmaceutically active agents, e.g. an mTOR inhibitor and an antifolate compound are packaged separately, but instruction for simultaneous or sequential administration are given.

In another aspect the present invention provides
1.3 A pharmaceutical composition comprising an mTOR inhibitor and an antifolate compound in combination beside pharmaceutically acceptable excipient.
1.4 A pharmaceutical package comprising an mTOR inhibitor and an antifolate compound beside instructions for combined administration.
1.5 A pharmaceutical package comprising an mTOR inhibitor beside instructions for combined administration with an antifolate compound.
1.6 A pharmaceutical package comprising an antifolate compound beside instructions for combined administration with an mTOR inhibitor.

A combination provided by the present invention includes a pharmaceutical combination, a pharmaceutical composition and a pharmaceutical package according to the present invention and is herein also designated as “A combination of (according to) the present invention”.

A combination of the present invention is useful for the treatment of disorders which are mediated by mTOR and/or thymidylate synthetase, e.g. disorders which are mediated by mTOR and thymidylate synthetase, such as disorders for which mTOR inhibitors and/or antifolate compounds, e.g. disorders for which mTOR inhibitors and antifolate compounds, are useful, e.g. for the treatment of inflammatory, immune disorders, e.g. autoimmune disorders, such as psoriasis and for the treatment of disorders associated with cancer, e.g. disorders associated with cell overproliferation, such as disorders associated with
cancer,
solid tumors,
endocrine tumors,
the growth of neoplasms,
mesothelioma.

Disorders associated with cancer, e.g. disorders associated with cell overproliferation, as used herein is meant to include e.g. premalignant conditions, hyperproliferative disorders, cancers whether primary or metastatic, cervical cancer, cancer originating from uncontrolled cellular proliferation, solid tumors, disorders associated with tumor growth, lymphoma, B-cell or T-cell lymphoma, benign tumors, benign dysproliferative disorders, renal carcinoma, esophageal cancer, stomach cancer, bladder cancer, breast cancer, colon cancer, lung cancer, melanoma, nasopharyngeal cancer, osteocarcinoma, ovarian cancer, uterine cancer; prostate cancer, skin cancer, mycosis fungoides, leukemia, tumor neovascularization, angiomas, myelodysplastic disorders, unresponsiveness to normal death-inducing signals (immortalization), increased cellular motility and invasiveness, genetic instability, dysregulated gene expression, (neuro)endocrine cancer (carcinoids), blood cancer, lymphocytic leukemias, neuroblastoma (neuroblastoma is a disease in which malignant (cancer) cells form in nerve tissue of the adrenal gland, neck, chest, or spinal cord), soft tissue cancer, metastasis,

Disorders associated with cancer as used herein is meant to include solid tumors and/or tumor metastasis (wherever located), e.g. brain and other central nervous system tumors (eg. tumors of the meninges, brain, spinal cord, cranial nerves and other parts of central nervous system, e.g. glioblastomas or medulla blastomas); head and/or neck cancer; breast tumors; circulatory system tumors (e.g. heart, mediastinum and pleura, and other intrathoracic organs, vascular tumors and tumor-associated vascular tissue); excretory system tumors (e.g. kidney, renal pelvis, ureter, bladder, other and unspecified urinary organs); gastrointestinal tract tumors (e.g. oesophagus, stomach, small intestine, colon, colorectal, rectosigmoid junction, rectum, anus and anal canal), tumors involving the liver and intrahepatic bile ducts, gall bladder, other and unspecified parts of biliary tract, pancreas, other and digestive organs); head and neck; oral cavity (lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (e.g. vulva, vagina, Cervixuteri, Corpus uteri, uterus, ovary, and other sites associated with female genital organs, placenta, penis, prostate, testis, and other sites associated with male genital organs); respiratory tract tumors (e.g. nasal cavity and middle ear, accessory sinuses, larynx, trachea, bronchus and lung, e.g. small cell lung cancer or non-small cell lung cancer); skeletal system tumors (e.g. bone and articular cartilage of limbs, bone articular cartilage and other sites); skin tumors (e.g. malignantmelanoma of the skin, non-melanoma skin cancer, basal cell carcinoma of skin, squamous cell carcinoma of skin, mesothelioma, Kaposi's sarcoma, mycosis fungoides); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

Disorders associated with cancer as used herein is meant to include endocrine tumors disorders mediated by endocrine tumors, including neuroendocrine tumors, such as pancreatic neuroendocrine tumors. Carcinoid tumors are neuroendocrine tumors and include carcinoid tumors arising from the foregut, e.g., bronchial or gastric carcinoid; midgut, e.g., small intestine or appendiceal carcinoid tumors; or hindgut, e.g. rectal carcinoid tumors; such as carcinoid tumors of the GI tract. Symptoms of carcinoid cancer include e.g. a carcinoid syndrome.

Disorders associated with cancer as used herein is meant to include disorders associated with the growth of neoplasms, e.g. including choriocarcinoma, leukemia, adenocarcinoma of the female breast, epidermid cancers of the head and neck, squamous or small-cell lung cancer, and various lymphosarcomas.

Disorders associated with cancer as used herein is meant to include (malignant) mesothelioma, which is a cancer of mesothelial cells, e.g. which cancer can developed in the pleura, (pleural mesothelioma), abdomen, e.g. mesothelioma of the tissues lining the abdominal cavity (peritoneal mesothelioma), and the lining of the lungs, or in the lining of the reproductive organs (begnin mesothelioma), recurrent non-small cell lung cancer (NSCLC), gestational trophoblastic tumor.

Where hereinbefore and subsequently a tumor, a tumor disease, a carcinoma or a cancer is mentioned, also metastasis in the original organ or tissue and/or in any other location are implied alternatively or in addition, whatever the location of the tumor and/or metastasis is. Disorders as used herein include diseases.

In another aspect the present invention provides
2.1 A method for the treatment of disorders which are mediated by mTOR and/or thymidylate synthetase,
such as inflammatory, (auto)immune, allergic disorders, or disorders associated with cancer, e.g. disorders associated with cell overproliferation;
preferably autoimmune disorders and disorders associated with cancer,
comprising administering to a subject in need thereof a therapeutically effective amount of an mTOR inhibitor in combination with an antifolate compound, e.g. either in sequence or simultaneously.
2.2 A method for the treatment of cancer, e.g. which cancer is mediated by mTOR and/or thymidylate synthetase, comprising administering to a subject in need thereof a therapeutically effective amount of an mTOR inhibitor in combination with an antifolate compound, e.g. either in sequence or simultaneously.
2.3 A method for inhibiting cancer growth, e.g. which cancer is mediated by mTOR and/or thymidylate synthetase, comprising administering to a subject in need thereof a therapeutically effective amount of an mTOR inhibitor in combination with an antifolate compound, e.g. either in sequence or simultaneously.
2.4 A method for inhibiting or controlling cancer, e.g. which cancer is mediated by mTOR and/or thymidylate synthetase, comprising administering to a subject in need thereof a therapeutically effective amount of an mTOR inhibitor in combination with an antifolate compound, e.g. either in sequence or simultaneously.
2.5 A method for inducing cancer regression, e.g. which cancer is mediated by mTOR and/or thymidylate synthetase, e.g. inducing cancer mass reduction, comprising administering to a subject in need thereof a therapeutically effective amount of an mTOR inhibitor in combination with an antifolate compound, e.g. either in sequence or simultaneously.
2.6 A method for treating cancer invasiveness or symptoms associated with such cancer growth, e.g. which cancer is mediated by mTOR and/or thymidylate synthetase, comprising administering to a subject in need thereof a therapeutically effective amount of an mTOR inhibitor in combination with an antifolate compound, e.g. either in sequence or simultaneously.
2.7 A method for preventing metastatic spread of cancer cells, e.g. which cancer is mediated by mTOR and/or thymidylate synthetase, comprising administering to a subject in need thereof a therapeutically effective amount of an mTOR inhibitor in combination with an antifolate compound, e.g. either in sequence or simultaneously.

Such treatment can be effected by use of any combination as set out under 1.1. to 1.6 above. In another aspect the present invention provides
3.1 The use of any combination as set out under 1.1. to 1.6 above for any method as set out under 2.1 to 2.7 above.
3.2 Any combination as set out under 1.1. to 1.6 above for any method as set out under 2.1 to 2.7 above.

In another aspect the present invention provides
4.1 The use of a combination of an mTOR inhibitor with an antifolate compound according to the present invention as a pharmaceutical, e.g. for use in any method as set out under 2.1 to 2.7 above.
4.2 A combination of an mTOR inhibitor with an antifolate compound according to the present invention as a pharmaceutical, e.g. for use in any method as set out under 2.1 to 2.7 above.

In another aspect the present invention provides
5.1 The use of a combination of an mTOR inhibitor with an antifolate compound according to the present invention for the manufacture of a medicament, e.g. for use in any method as set out under 2.1 to 2.7 above.
5.2 A combination of an mTOR inhibitor with an antifolate compound according to the present invention for use in the preparation of a medicament, e.g. for use in any method as set out under 2.1 to 2.7 above.
5.3 A method for the preparation of a medicament for use in any method as set out under 2.1 to 2.7 above, comprising either
   a) combining, e.g. mixing, an mTOR inhibitor and an antifolate compound with pharmaceutically acceptable excipient, or
   b) combining e.g. mixing, an mTOR inhibitor with pharmaceutically acceptable excipient to obtain a pharmaceutical composition COMP1, and combining, e.g. mixing, an antifolate compound, with pharmaceutically acceptable excipient to obtain a pharmaceutical composition COMP2, and either
      (i) combining the pharmaceutical composition COMP1 and the pharmaceutical composition COMP2 in one single package, or
      (ii) packing the pharmaceutical composition COMP1 separately and packing the pharmaceutical composition COMP2 separately, but adding to each package instructions for combined administration of pharmaceutical composition COMP1 and pharmaceutical composition COMP2.

Combined treatment of disorders which are mediated by mTOR and/or thymidylate synthetase according to the present invention may provide improvements compared with single treatment, e.g. toxicity of antifolate compounds may be lowered, the activity of an mTOR inhibitor or the activity of an antifolate compound may be increased compared with single treatment, e.g. combined treatment may result in synergistic effects or may overcome resistance against an mTOR inhibitor or an antifolate compound, e.g. when used in any method as set out under 2.1 to 2.7 above.

In another aspect the present invention provides
6.1 A combination according to the present invention comprising an amount of an mTOR inhibitor and an amount of an antifolate compound, wherein the amounts are appropriate to produce a synergistic therapeutic effect.
6.2—A method for improving the therapeutic utility of an antifolate compound comprising co-administering, e.g. concomitantly or in sequence, a therapeutically effective amount of an mTOR inhibitor and an antifolate compound.
6.3 A method for improving the therapeutic utility of an mTOR inhibitor comprising co-administering, e.g. concomitantly or in sequence, a therapeutically effective amount of an mTOR inhibitor and an antifolate compound.
6.4 A method of reducing the toxicity associated with the administration of an antifolate compound, comprising administering to said subject an effective amount of an antifolate compound in combination with an effective amount of an mTOR inhibitor.
   e.g. for use in any method as set out under 2.1 to 2.7 above.

Treatment as used herein includes treatment and prevention, preferably treatment.

A combination according to the present invention, e.g. for use in any method provided according to the present invention, may further comprise another drug substance.

Another drug substance as used herein includes any drug other than an mTOR inhibitor or an antifolate compound which may have beneficial effects in a use or a method provided by the present invention. Such drugs e.g. include
anti-inflammatory and/or immunomodulatory drugs,
anticancer drugs
anesthetic drugs
antidiarrheal drugs.

In another aspect the present invention provides
7.1 A combination according to the present invention, a method or a use provided by the present invention comprising beside a combination of an mTOR inhibitor with an antifolate compound at least one other drug substance,
e.g. wherein the other drug substance is selected from an anti-inflammatory drug, an immunomodulatory drug, an anticancer drug, an anesthetic drug, and/or an antidiarrheal drug.

In another aspect the present invention provides the use of an mTOR inhibitor in combination with an antifolate compound in any of a method provided by the present invention.

In another aspect the present invention provides the use of an mTOR inhibitor in combination with an antifolate compound, in any of a combination or pharmaceutical composition, or pharmaceutical package provided by the present invention.

Anti-inflammatory and/or immunomodulatory drugs which are prone to be useful in combination, e.g. in combination therapy, with a combination of the present invention include e.g.
mediators, e.g. inhibitors, of calcineurin, e.g. cyclosporin A, FK 506;
ascomycins having immuno-suppressive properties, e.g. ABT-281, ASM981;
corticosteroids; cyclophosphamide; azathioprene; leflunomide; mizoribine;
mycophenolic acid or salt; e.g. sodium, mycophenolate mofetil;
15-deoxyspergualine or an immunosuppressive homologue, analogue or derivative thereof;
mediators, e.g. inhibitors, of bcr-abl tyrosine kinase activity;
mediators, e.g. inhibitors, of c-kit receptor tyrosine kinase activity;
mediators, e.g. inhibitors, of PDGF receptor tyrosine kinase activity, e.g. Gleevec (imatinib);
mediators, e.g. inhibitors, of p38 MAP kinase activity,
mediators, e.g. inhibitors, of VEGF receptor tyrosine kinase activity,
mediators, e.g. inhibitors, of PKC activity, e.g. as disclosed in WO0238561 or WO0382859, e.g. the compound of Example 56 or 70;
mediators, e.g. inhibitors, of JAK3 kinase activity, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO2004052359 or WO2005066156;
mediators, e.g. agonists or modulators of S1P receptor activity, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts;
immunosuppressive monoclonal antibodies, e.g., monoclonal antibodies to leukocyte receptors, e.g., Blys/BAFF receptor, MHC, CD2, CD3, CD4, CD7, CD8, CD25, CD28, CD40, CD45, CD52, CD58, CD80, CD86, IL-12 receptor, IL-17 receptor, IL-23 receptor or their ligands;
other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y;
mediators, e.g. inhibitors of adhesion molecule activities, e.g. LFA-1 antagonists, ICAM-1 or -3 antagonists, VCAM-4 antagonists or VLA-4 antagonists,
mediators, e.g. inhibitors, of MIF activity,
5-aminosalicylate (5-ASA) agents, such as sulfasalazine, Azulfidine®, Asacol®, Dipentum®, Pentasa®, Rowasa®, Canasa®, Colazal®, e.g. drugs containing mesalamine; e.g mesalazine in combination with heparin;
mediators, e.g. inhibitors, of TNF-alpha activity, e.g. including antibodies which bind to TNF-alpha, e.g. infliximab (Remicade®), thalidomide, lenalidomide,
nitric oxide releasing non-steriodal anti-inflammatory drugs (NSAIDs), e.g. including COX-inhibiting NO-donating drugs (CINOD);
phosphordiesterase, e.g. mediators, such as inhibitors of PDE4B activity,
mediators, e.g. inhibitors, of caspase activity,
mediators, e.g. agonists, of the G protein coupled receptor GPBAR1,
mediators, e.g. inhibitors, of ceramide kinase activity,
‘multi-functional anti-inflammatory’ drugs (MFAIDs), e.g. cytosolic phospholipase A2 (cPLA2) inhibitors, such as membrane-anchored phospholipase A2 inhibitors linked to glycosaminoglycans;
antibiotics, such as penicillins, cephalosporins, erythromycins, tetracyclines, sulfonamides, such as sulfadiazine, sulfisoxazole; sulfones, such as dapsone; pleuromutilins, fluoroquinolones, e.g. metronidazole, quinolones such as ciprofloxacin; levofloxacin; probiotics and commensal bacteria e.g. Lactobacillus, Lactobacillus reuteri;
antiviral drugs, such as ribivirin, vidarabine, acyclovir, ganciclovir, zanamivir, oseltamivir phosphate, famciclovir, atazanavir, amantadine, didanosine, efavirenz, foscarnet, indinavir, lamivudine, nelfinavir, ritonavir, saquinavir, stavudine, valacyclovir, valganciclovir, zidovudine.

Anti-inflammatory drugs which are prone to be useful in combination, e.g. in combination therapy, with a combination of the present invention include e.g. non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); cyclooxygenase-2 (COX-2) inhibitors such as celecoxib; inhibitors of phosphodiesterase type IV (PDE-IV); antagonists of the chemokine receptors, especially CCR1, CCR2, and CCR3; cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; anticholinergic agents such as muscarinic antagonists (ipratropium bromide); other compounds such as theophylline, sulfasalazine and aminosalicylates, e.g. 5-aminosalicylic acid and prodrugs thereof, antirheumatics.

Anticancer drugs which are prone to be useful as a combination partner, e.g. in combination therapy, with a combination of the present invention, e.g. include
i. a steroid; e.g. prednisone.
ii. an adenosine-kinase-inhibitor; which targets, decreases or inhibits nucleobase, nucleoside, nucleotide and nucleic acid metabolisms, such as 5-Iodotubercidin, which is also known as 7H-pyrrolo[2,3-d]pyrimidin-4-amine, 5-iodo-7-β-D-ribofuranosyl.
iii. an adjuvant; which enhances the 5-FU-TS bond as well as a compound which targets, decreases or inhibits, alkaline phosphatase, such as leucovorin, levamisole.
iv. an adrenal cortex antagonist; which targets, decreases or inhibits the activity of the adrenal cortex and changes the peripheral metabolism of corticosteroids, resulting in a decrease in 17-hydroxycorticosteroids, such as mitotane.
v. an AKT pathway inhibitor; such as a compound which targets, decreases or inhibits Akt, also known as protein kinase B (PKB), such as deguelin, which is also known as 3H-bis[1]benzopyrano[3,4-b:6′,5′-e]pyran-7(7aH)-one, 13,13a-dihydro-9,10-dimethoxy-3,3-dimethyl-, (7aS,13aS); and triciribine, which is also known as 1,4,5,6,8-pentaazaacenaphthylen-3-amine, 1,5-dihydro-5-methyl-1-β-D-ribofuranosyl.
vi. an alkylating agent; which causes alkylation of DNA and results in breaks in the DNA molecules as well as cross-linking of the twin strands, thus interfering with DNA replication and transcription of RNA, such as nitrogen mustards, e.g. chlorambucil, chlormethine, cyclophosphamide, ifosfamide, melphalan, estramustine (Emcyt®); nitrosueras, such as carmustine, fotemustine, lomustine, streptozocin (streptozotocin, STZ, Zanosar®), BCNU; Gliadel; dacarbazine, mechlorethamine, e.g. in the form of a hydrochloride, procarbazine, e.g. in the form of a hydrochloride, thiotepa, temozolomide (TEMODAR®), mitomycin, altretamine, busulfan, estramustine, uramustine. Cyclophosphamide can be administered, e.g., in the form as it is marketed, e.g., under the trademark CYCLOSTIN®; and ifosfamide as HOLOXAN®.
vii. an angiogenesis inhibitor; which targets, decreases or inhibits the production of new blood vessels, e.g. which targets methionine aminopeptidase-2 (MetAP-2), macrophage inflammatory protein-1 (MIP-1alpha), CCL5, TGF-beta, lipoxygenase, cyclooxygenase, and topoisomerase, or which indirectly targets p21, p53, CDK2 and collagen synthesis, e.g. including fumagillin, which is known as 2,4,6,8-decatetraenedioic acid, mono[(3R,4S,5S,6R)-5-methoxy-4-[(2R,3R)-2-methyl-3-(3-methyl-2-butenyl)oxiranyl]-1-oxaspiro[2.5]oct-6-yl] ester, (2E,4E,6E,8E)-(9CI); shikonin, which is also known as 1,4-naphthalenedione, 5,8-dihydroxy-2-[(1R)-1-hydroxy-4-methyl-3-pentenyl]-(9CI); tranilast, which is also known as benzoic acid, 2-[[3-(3,4-dimethoxyphenyl)-1-oxo-2-propenyl]amino]; ursolic acid; suramin; bengamide or a derivative thereof, thalidomide, TNP-470.
viii. an anti-androgen; which blocks the action of androgens of adrenal and testicular origin which stimulate the growth of normal and malignant prostatic tissue, such as nilutamide; bicalutamide (CASODEX®), which can be formulated, e.g., as disclosed in U.S. Pat. No. 4,636,505.
ix. an anti-estrogen; which antagonizes the effect of estrogens at the estrogen receptor level, e.g. including an aromatase inhibitor, which inhibits the estrogen production, i.e. the conversion of the substrates androstenedione and testosterone to estrone and estradiol, respectively,
e.g. including atamestane, exemestane, formestane, aminoglutethimide, roglethimide, pyridoglutethimide, trilostane, testolactone, ketokonazole, vorozole, fadrozole, anastrozole, letrozole, toremifene; bicalutamide; flutamide; tamoxifen, tamoxifen citrate; tamoxifen; fulvestrant; raloxifene, raloxifene hydrochloride. Tamoxifen may be e.g. administered in the form as it is marketed, e.g., NOLVADEX®; and raloxifene hydrochloride is marketed as EVISTA®. Fulvestrant may be formulated as disclosed in U.S. Pat. No. 4,659,516 and is marketed as FASLODEX®.
x. an anti-hypercalcemia agent; which is used to treat hypercalcemia, such as gallium (III) nitrate hydrate; and pamidronate disodium.
xi. an antimetabolite; which inhibits or disrupts the synthesis of DNA resulting in cell death, such as folic acids, e.g. methotrexate, permetrexed, raltitrexed; purins, e.g. 6-mercaptopurine, cladribine, clofarabine; fludarabine, thioguanine (tioguanine), 6-thioguanine, pentostatin (deoxycoformycin); cytarabine; flexuridine; fluorouracil; 5-fluorouracil (5-FU), floxuridine (5-FUdR), capecitabine; gemcitabine; gemcitabine hydrochloride; hydroxyurea (e.g. Hydrea®); DNA de-methylating agents, such as 5-azacytidine and decitabine; edatrexate. Capecitabine and gemcitabine can be administered e.g. in the marketed form, such as XELODA® and GEMZAR®.
xii. an apoptosis inducer; which induces the normal series of events in a cell that leads to its death, e.g. selectively inducing the X-linked mammalian inhibitor of apoptosis protein XIAP, or e.g. downregulating BCL-xL; such as ethanol, 2-[[3-(2,3-dichlorophenoxy)propyl]amino]; gambogic acid; embelin, which is also known as 2,5-cyclohexadiene-1,4-dione, 2,5-dihydroxy-3-undecyl-(9CI); arsenic trioxide.
xiii. an aurora kinase inhibitor; which targets, decreases or inhibits later stages of the cell cycle from the G2/M check point all the way through to the mitotic checkpoint and late mitosis; such as binucleine 2, which is also known as methanimidamide, N′-[1-(3-chloro-4-fluorophenyl)-4-cyano-1H-pyrazol-5-yl]-N,N-dimethyl-(9CI).
xiv. a Bruton's Tyrosine Kinase (BTK) inhibitor; which targets, decreases or inhibits human and murine B cell development; such as terreic acid.
xv. a calcineurin inhibitor; which targets, decreases or inhibits the T cell activation pathway, such as cypermethrin, which is also known as cyclopropanecarboxylic acid, 3-(2,2-dichloroethenyl)-2,2-dimethyl-,cyano(3-phenoxyphenyl)methyl ester; deltamethrin, which is also known as cyclopropanecarboxylic acid, 3-(2,2-dibromoethenyl)-2,2-dimethyl-(S)-cyano(3-phenoxyphenyl)methyl ester, (1R,3R); fenvalerate, which is also known as benzeneacetic acid, 4-chloro-α-(1-methylethyl)-,cyano(3-phenoxyphenyl)methyl ester; and Tyrphostin 8; but excluding cyclosporin or FK506.
xvi. a CaM kinase II inhibitor; which targets, decreases or inhibits CaM kinases; constituting a family of structurally related enzymes that include phosphorylase kinase, myosin light chain kinase, and CaM kinases I-IV; such as 5-isoquinolinesulfonic acid, 4-[(2S)-2-[(5-isoquinolinylsulfonyl)methylamino]-3-oxo-3-(4-phenyl-1-piperazinyl)propyl]phenyl ester (9CI); benzenesulfonamide, N-[2-[[[3-(4-chlorophenyl)-2-propenyl]methyl]amino]methyl]phenyl]-N-(2-hydroxyethyl)-4-methoxy.
xvii. a CD45 tyrosine phosphatase inhibitor; which targets, decreases or inhibits dephosphorylating regulatory pTyr residues on Src-family protein-tyrosine kinases, which aids in the treatment of a variety of inflammatory and immune disorders; such as phosphonic acid, [[2-(4-bromophenoxy)-5-nitrophenyl]hydroxymethyl].
xviii. a CDC25 phosphatase inhibitor; which targets, decreases or inhibits overexpressed dephosphorylate cyclin-dependent kinases in tumors; such as 1,4-naphthalenedione, 2,3-bis[(2-hydroyethyl)thio].
xix. a CHK kinase inhibitor; which targets, decreases or inhibits overexpression of the antiapoptotic protein Bcl-2; such as debromohymenialdisine. Targets of a CHK kinase inhibitor are CHK1 and/or CHK2.
xx. a controlling agent for regulating genistein, olomucine and/or tyrphostins; such as daidzein, which is also known as 4H-1-benzopyran-4-one, 7-hydroxy-3-(4-hydroxyphenyl); Iso-Olomoucine, and Tyrphostin 1.
xxi. a cyclooxygenase inhibitor; e.g. including Cox-2 inhibitors; which targets, decreases or inhibits the enzyme Cox-2 (cyclooxygenase-2); such as 1H-indole-3-acetamide, 1-(4-chlorobenzoyl)-5-methoxy-2-methyl-N-(2-phenylethyl); 5-alkyl substituted 2-arylaminophenylacetic acid and derivatives, e.g. celecoxib (CELEBREX®), rofecoxib (VIOXX®), etoricoxib, valdecoxib; or a 5-alkyl-2-arylaminophenylacetic acid, e.g., 5-methyl-2-(2′-chloro-6′-fluoroanilino)phenyl acetic acid, lumiracoxib; and celecoxib.
xxii. a cRAF kinase inhibitor; which targets, decreases or inhibits the up-regulation of E-selectin and vascular adhesion molecule-1 induced by TNF; such as 3-(3,5-dibromo-4-hydroxybenzylidene)-5-iodo-1,3-dihydroindol-2-one; and benzamide, 3-(dimethylamino)-N-[3-[(4-hydroxybenzoyl)amino]-4-methylphenyl]. Raf kinases play an important role as extracellular signal-regulating kinases in cell differentiation, proliferation, and apoptosis. A target of a cRAF kinase inhibitor includes, but is not limited, to RAF1.
xxiii. a cyclin dependent kinase inhibitor; which targets, decreases or inhibits cyclin dependent kinase playing a role in the regulation of the mammalian cell cycle; such as N9-isopropyl-olomoucine; olomoucine; purvalanol B, which is also known as Benzoic acid, 2-chloro-4-[[2-[[(1R)-1-(hydroxymethyl)-2-methylpropyl]amino]-9-(1-methylethyl)-9H-purin-6-yl]amino]-(9CI); roascovitine; indirubin, which is also known as 2H-indol-2-one, 3-(1,3-dihydro-3-oxo-2H-indol-2-ylidene)-1,3-dihydro-(9CI); kenpaullone, which is also known as indolo[3,2-d][1]benzazepin-6(5H)-one, 9-bromo-7,12-dihydro-(9CI); purvalanol A, which is also known as 1-Butanol, 2-[[6-[(3-chlorophenyl)amino]-9-(1-methylethyl)-9H-purin-2-yl]amino]-3-methyl-, (2R)-(9CI); indirubin-3′-monooxime. Cell cycle progression is regulated by a series of sequential events that include the activation and subsequent inactivation of cyclin dependent kinases (Cdks) and cyclins. Cdks are a group of serine/threonine kinases that form active heterodimeric complexes by binding to their regulatory subunits, cyclins. Examples of targets of a cyclin dependent kinase inhibitor include, but are not limited to, CDK, AHR, CDK1, CDK2, CDK5, CDK4/6, GSK3beta, and ERK.
xxiv. a cysteine protease inhibitor; which targets, decreases or inhibits cystein protease which plays a vital role in mammalian cellular turnover and apotosis; such as 4-morpholinecarboxamide,N-[(1S)-3-fluoro-2-oxo-1-(2-phenylethyl)propyl]amino]-2-oxo-1-(phenylmethyl)ethyl].
xxv. a DNA intercalator; which binds to DNA and inhibits DNA, RNA, and protein synthesis; such as plicamycin, dactinomycin.
xxvi. a DNA strand breaker; which causes DNA strand scission and results in inhibition of DNA synthesis, inhibition of RNA and protein synthesis; such as bleomycin.
xxvii. an E3 Ligase inhibitor; which targets, decreases or inhibits the E3 ligase which inhibits the transfer of ubiquitin chains to proteins, marking them for degradation in the proteasome; such as N-((3,3,3-trifluoro-2-trifluoromethyl)propionyl)sulfanilamide.
xxviii. an endocrine hormone; which by acting mainly on the pituitary gland causes the suppression of hormones in males, the net effect being a reduction of testosterone to castration levels; in females, both ovarian estrogen and androgen synthesis being inhibited; such as leuprolide; megestrol, megestrol acetate.
xxix. compounds targeting, decreasing or inhibiting the activity of the epidermal growth factor family of receptor tyrosine kinases (EGFR, ErbB2, ErbB3, ErbB4 as homo- or heterodimers), such as compounds, proteins or antibodies which inhibit members of the EGF receptor tyrosine kinase family, e.g. EGF receptor, ErbB1, ErbB2, ErbB3 and ErbB4 or bind to EGF or EGF-related ligands, and are in particular those compounds, proteins or monoclonal antibodies generically and specifically disclosed in WO 9702266, e.g. the compound of ex. 39, EP0564409, WO9903854, EP0520722, EP0566226, EP0787722, EP0837063, U.S. Pat. No. 5,747,498, WO9810767, WO9730034, WO9749688, WO9738983 and, especially, WO9630347, e.g. a compound known as CP 358774, WO9633980, e.g. a compound known as ZD 1839; and WO 9503283, e.g. a compound known as ZM105180, e.g including the dual acting tyrosine kinase inhibitor (ErbB1 and ErbB2) lapatinib (GSK572016), e.g. lapatinib ditosylate; panituzumab, trastuzumab (HERCEPTIN®), cetuximab, iressa, OSI-774, CI-1033, EKB-569, GW-2016, E1.1, E2.4, E2.5, E6.2, E6.4, E2.11, E6.3 or E7.6.3, 7H-pyrrolo-[2,3-d]pyrimidine derivatives which are e.g. disclosed in WO03013541, erlotinib, gefitinib. Erlotinib can be administered in the form as it is marketed, e.g. TARCEVA®, and gefitinib as IRESSA®, human monoclonal antibodies against the epidermal growth factor receptor including ABX-EGFR.
xxx. an EGFR, PDGFR tyrosine kinase inhibitor; such as EGFR kinase inhibitors including tyrphostin 23, tyrphostin 25, tyrphostin 47, tyrphostin 51 and tyrphostin AG 825; 2-propenamide, 2-cyano-3-(3,4-dihydroxyphenyl)-N-phenyl-(2E); tyrphostin Ag 1478; lavendustin A; 3-pyridineacetonitrile, α-[(3,5-dichlorophenyl)methylene]-, (αZ); an example of an EGFR, PDGFR tyrosine kinase inhibitor e.g. includes tyrphostin 46. PDGFR tyrosine kinase inhibitor including tyrphostin 46. Targets of an EGFR kinase inhibitor include guanylyl cyclase (GC-C) HER2, EGFR, PTK and tubulin.
xxxi. a farnesyltransferase inhibitor; which targets, decreases or inhibits the Ras protein; such as a-hydroxyfarnesylphosphonic acid; butanoic acid, 2-[[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-,1-methylethyl ester, (2S); manumycin A; L-744,832 or DK8G557, tipifarnib (R115777), SCH66336 (lonafarnib), BMS-214662,
xxxii. a Flk-1 kinase inhibitor; which targets, decreases or inhibits Flk-1 tyrosine kinase activity; such as 2-propenamide, 2-cyano-3-[4-hydroxy-3,5-bis(1-methylethyl)phenyl]-N-(3-phenylpropyl)-(2E). A target of a Flk-1 kinase inhibitor includes, but is not limited to, KDR.
xxxiii. a Glycogen synthase kinase-3 (GSK3) inhibitor; which targets, decreases or inhibits glycogen synthase kinase-3 (GSK3); such as indirubin-3′-monooxime. Glycogen Synthase Kinase-3 (GSK-3; tau protein kinase I), a highly conserved, ubiquitously expressed serine/threonine protein kinase, is involved in the signal transduction cascades of multiple cellular processes. which is a protein kinase that has been shown to be involved in the regulation of a diverse array of cellular functions, including protein synthesis, cell proliferation, cell differentiation, microtubule assembly/disassembly, and apoptosis.
xxxiv. a histone deacetylase (HDAC) inhibitor; which inhibits the histone deacetylase and which possess anti-proliferative activity; such as compounds disclosed in WO0222577, especially N-hydroxy-3-[4-[[(2-hydroxyethyl)[2-(1H-indol-3-yl)ethyl]-amino]methyl]phenyl]-2E-2-propenamide, and N-hydroxy-3-[4-[[[2-(2-methyl-1H-indol-3-yl)-ethyl]-amino]methyl]phenyl]-2E-2-propenamide and pharmaceutically acceptable salts thereof; suberoylanilide hydroxamic acid (SAHA); [4-(2-amino-phenylcarbamoyl)-benzyl]-carbamic acid pyridine-3-ylmethyl ester and derivatives thereof; butyric acid, pyroxamide, trichostatin A, oxamflatin, apicidin, depsipeptide; depudecin; trapoxin, HC toxin, which is also known as cyclo[L-alanyl-D-alanyl-(□S,2S)-□-amino-□-oxooxiraneoctanoyl-D-prolyl] (9CI); sodium phenylbutyrate, suberoyl bis-hydroxamic acid; Trichostatin A, BMS-27275, pyroxamide, FR-901228, valproic acid.
xxxv. a HSP90 inhibitor; which targets, decreases or inhibits the intrinsic ATPase activity of HSP90; degrades, targets, decreases or inhibits the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, e.g., 17-allylamino, 17-demethoxygeldanamycin (17AAG), a geldanamycin derivative; other geldanamycin-related compounds; radicicol and HDAC inhibitors. Other examples of an HSP90 inhibitor include geldanamycin, 17-demethoxy-17-(2-propenylamino). Potential indirect targets of an HSP90 inhibitor include FLT3, BCR-ABL, CHK1, CYP3A5*3 and/or NQ01*2. Nilotinib is an example of an BCR-ABL tyrosine kinase inhibitor.
xxxvi. a I-kappa B-alpha kinase inhibitor (IKK); which targets, decreases or inhibits NF-kappaB, such as 2-propenenitrile, 3-[(4-methylphenyl)sulfonyl]-(2E).
xxxvii. an insulin receptor tyrosine kinase inhibitor; which modulates the activities of phosphatidylinositol 3-kinase, microtubule-associated protein, and S6 kinases; such as hydroxyl-2-naphthalenylmethylphosphonic acid, LY294002.
xxxviii. a c-Jun N-terminal kinase (JNK) kinase inhibitor; which targets, decreases or inhibits Jun N-terminal kinase; such as pyrazoleanthrone and/or epigallocatechin gallate. Jun N-terminal kinase (JNK), a serine-directed protein kinase, is involved in the phosphorylation and activation of c-Jun and ATF2 and plays a significant role in metabolism, growth, cell differentiation, and apoptosis. A target for a JNK kinase inhibitor includes, but is not limited to, DNMT.
xxxix a microtubule binding agent; which acts by disrupting the microtubular network that is essential for mitotic and interphase cellular function; such as vinca alkaloids, e.g. vinblastine, vinblastine sulfate; vincristine, vincristine sulfate; vindesine; vinorelbine; taxanes, such as taxanes, e.g. docetaxel; paclitaxel; discodermolides; cochicine, epothilones and derivatives thereof, e.g. epothilone B or a derivative thereof. Paclitaxel is marketed as TAXOL®; docetaxel as TAXOTERE®; vinblastine sulfate as VINBLASTIN R.P®; and vincristine sulfate as FARMISTIN®. Also included are the generic forms of paclitaxel as well as various dosage forms of paclitaxel. Generic forms of paclitaxel include, but are not limited to, betaxolol hydrochloride. Various dosage forms of paclitaxel include, but are not limited to albumin nanoparticle paclitaxel marketed as ABRAXANE®; ONXOL®, CYTOTAX®. Discodermolide can be obtained, e.g., as disclosed in U.S. Pat. No. 5,010,099. Also included are Epotholine derivatives which are disclosed in U.S. Pat. No. 6,194,181, WO98/0121, WO9825929, WO9808849, WO9943653, WO9822461 and WO0031247. Especially preferred are Epotholine A and/or B.
xl. a mitogen-activated protein (MAP) kinase-inhibitor; which targets, decreases or inhibits Mitogen-activated protein, such as benzenesulfonamide, N-[2-[[[3-(4-chlorophenyl)-2-propenyl]methyl]amino]methyl]phenyl]-N-(2-hydroxyethyl)-4-methoxy. The mitogen-activated protein (MAP) kinases are a group of protein serine/threonine kinases that are activated in response to a variety of extracellular stimuli and mediate signal transduction from the cell surface to the nucleus. They regulate several physiological and pathological cellular phenomena, including inflammation, apoptotic cell death, oncogenic transformation, tumor cell invasion, and metastasis.
xli. a MDM2 inhibitor; which targets, decreases or inhibits the interaction of MDM2 and the p53 tumor suppressor; such as trans-4-iodo, 4′-boranyl-chalcone.
xlii. a MEK inhibitor; which targets, decreases or inhibits the kinase activity of MAP kinase MEK; such as sorafenib, e.g. Nexavar® (sorafenib tosylate), butanedinitrile, bis[amino[2-aminophenyl)thio]methylene]. A target of a MEK inhibitor includes, but is not limited to ERK. An indirect target of a MEK inhibitor includes, but is not limited to, cyclin D1.
xliii: a matrix metalloproteinase inhibitor (MMP) inhibitor; which targets, decreases or inhibits a class of protease enzyme that selectively catalyze the hydrolysis of polypeptide bonds including the enzymes MMP-2 and MMP-9 that are involved in promoting the loss of tissue structure around tumors and facilitating tumor growth, angiogenesis, and metastasis such as actinonin, which is also known as butanediamide, N-4-hydroxy-N1-[(1S)-1-[[(2S)-2-(hydroxymethyl)-1-pyrrolidinyl]carbonyl]-2-methylpropyl]-2-pentyl-, (2R)-(9CI); epigallocatechin gallate; collagen peptidomimetic and non-peptidomimetic inhibitors; tetracycline derivatives, e.g., hydroxamate peptidomimetic inhibitor batimastat; and its orally-bioavailable analogue marimastat, prinomastat, metastat, neovastat, tanomastat, TAA211, BMS-279251, BAY 12-9566, MMI270B or AAJ996. A target of a MMP inhibitor includes, but is not limited to, polypeptide deformylase.
xliv. a NGFR tyrosine-kinase-inhibitor; which targets, decreases or inhibits nerve growth factor dependent p140^{c-trk} tyrosine phosphorylation; such as tyrphostin AG 879. Targets of a NGFR tyrosine-kinase-inhibitor include, but are not limited to, HER2, FLK1, FAK, TrkA, and/or TrkC. An indirect target inhibits expression of RAF1.
xlv. a p38 MAP kinase inhibitor, including a SAPK2/p38 kinase inhibitor; which targets, decreases or inhibits p38-MAPK, which is a MAPK family member, such as phenol, 4-[4-(4-fluorophenyl)-5-(4-pyridinyl)-1H-imidazol-2-yl]. An example of a SAPK2/p38 kinase inhibitor includes, but is not limited to, benzamide, 3-(dimethylamino)-N-[3-[(4-hydroxybenzoyl)amino]-4-methylphenyl]. A MAPK family member is a serine/threonine kinase activated by phosphorylation of tyrosine and threonine residues. This kinase is phosphorylated and activated by many cellular stresses and inflammatory stimuli, thought to be involved in the regulation of important cellular responses such as apoptosis and inflammatory reactions.
xlvi. a p56 tyrosine kinase inhibitor; which targets, decreases or inhibits p56 tyrosine kinase, which is an enzyme that is a lymphoid-specific src family tyrosine kinase critical for T-cell development and activation; such as damnacanthal, which is also known as 2-anthracenecarboxaldehyde,9,10-dihydro-3-hydroxy-1 methoxy-9,10-dioxo, Tyrphostin 46. A target of a p56 tyrosine kinase inhibitor includes, but is not limited to, Lck. Lck is associated with the cytoplasmic domains of CD4, CD8 and the beta-chain of the IL-2 receptor, and is thought to be involved in the earliest steps of TCR-mediated T-cell activation.
xlvii. a PDGFR tyrosine kinase inhibitor; targeting, decreasing or inhibiting the activity of the C-kit receptor tyrosine kinases (part of the PDGFR family), such as targeting, decreasing or inhibiting the activity of the c-Kit receptor tyrosine kinase family, especially inhibiting the c-Kit receptor. Examples of targets of a PDGFR tyrosine kinase inhibitor includes, but are not limited to PDGFR, FLT3 and/or c-KIT; such as tyrphostin AG 1296; tyrphostin 9; 1,3-butadiene-1,1,3-tricarbonitrile,2-amino-4-(1H-indol-5-yl); N-phenyl-2-pyrimidine-amine derivative, e.g. imatinib, IRESSA®. PDGF plays a central role in regulating cell proliferation, chemotaxis, and survival in normal cells as well as in various disease states such as cancer, atherosclerosis, and fibrotic disease. The PDGF family is composed of dimeric isoforms (PDGF-M, PDGF-BB, PDGF-AB, PDGF-CC, and PDGF-DD), which exert their cellular effects by differentially binding to two receptor tyrosine kinases. PDGFR-α and PDGFR-β have molecular masses of −170 and 180 kDa, respectively.
xlviii. a phosphatidylinositol 3-kinase inhibitor; which targets, decreases or inhibits PI 3-kinase; such as wortmannin, which is also known as 3H-Furo[4,3,2-de]indeno[4,5-h]-2-benzopyran-3,6,9-trione, 11-(acetyloxy)-1,6b,7,8,9a,10,11,11b-octahydro-1-(methoxymethyl)-9a,11b-dimethyl-, (1S,6bR,9aS,11R,11bR)-(9CI); 8-phenyl-2-(morpholin-4-yl)-chromen-4-one; quercetin, quercetin dihydrate. PI 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation. An example of a target of a phosphatidylinositol 3-kinase inhibitor includes, but is not limited to, Pi3K.
xlix. a phosphatase inhibitor; which targets, decreases or inhibits phosphatase; such as cantharidic acid; cantharidin; and L-leucinamide, N-[4-(2-carboxyethenyl)benzoyl]glycyl-L-α-glutamyl-(E). Phosphatases remove the phosphoryl group and restore the protein to its original dephosphorylated state. Hence, the phosphorylation-dephosphorylation cycle can be regarded as a molecular “on-off” switch.
l. platinum agent; which contains platinum and inhibit DNA synthesis by forming interstrand and intrastrand cross-linking of DNA molecules; such as carboplatin; cisplatin; oxaliplatin; cisplatinum; satraplatin and platinum agents such as ZD0473, BBR3464. Carboplatin can be administered, e.g., in the form as it is marketed, e.g. CARBOPLAT®; and oxaliplatin as ELOXATIN®.
li. a protein phosphatase inhibitor, including a PP1 and PP2 inhibitor and a tyrosine phosphatase inhibitor; which targets, decreases or inhibits protein phosphatase. Examples of a PP1 and PP2A inhibitor include cantharidic acid and/or cantharidin. Examples of a tyrosine phosphatase inhibitor include, but are not limited to, L-P-bromotetramisole oxalate; 2(5H)-furanone,4-hydroxy-5-(hydroxymethyl)-3-(1-oxohexadecyl)-, (5R); and benzylphosphonic acid.
The term “a PP1 or PP2 inhibitor”, as used herein, relates to a compound which targets, decreases or inhibits Ser/Thr protein phosphatases. Type I phosphatases, which include PP1, can be inhibited by two heat-stable proteins known as Inhibitor-1 (I-1) and Inhibitor-2 (I-2). They preferentially dephosphorylate a subunit of phosphorylase kinase. Type II phosphatases are subdivided into spontaneously active (PP2A), CA²⁺-dependent (PP2B), and Mg²⁺-dependent (PP2C) classes of phosphatases.
The term “tyrosine phosphatase inhibitor”, as used here, relates to a compounds which targets, decreases or inhibits tyrosine phosphatase. Protein tyrosine phosphatases (PTPs) are relatively recent additions to the phosphatase family. They remove phosphate groups from phosphorylated tyrosine residues of proteins. PTPs display diverse structural features and play important roles in the regulation of cell proliferation, differentiation, cell adhesion and motility, and cytoskeletal function. Examples of targets of a tyrosine phosphatase inhibitor include, but are not limited to, alkaline phosphatase (ALP), heparanase, PTPase, and/or prostatic acid phosphatase.
lii. a PKC inhibitor and a PKC delta kinase inhibitor: The term “a PKC inhibitor”, as used herein, relates to a compound which targets, decreases or inhibits protein kinase C as well as its isozymes. Protein kinase C (PKC), a ubiquitous, phospholipid-dependent enzyme, is involved in signal transduction associated with cell proliferation, differentiation, and apoptosis. Examples of a target of a PKC inhibitor include, but are not limited to, MAPK and/or NF-kappaB. Examples of a PKC inhibitor include, but are not limited to, 1-H-pyrrolo-2,5-dione,3-[1-[3-(dimethylamino)propyl]-1H-indol-3-yl]-4-(1H-indol-3-yl); bisindolylmaleimide IX; sphingosine, which is known as 4-octadecene-1,3-diol, 2-amino-, (2S,3R,4E)-(9CI); staurosporine, which is known as 9,13-Epoxy-1H,9H-diindolo[1,2,3-gh:3′,2′,1′-Im]pyrrolo[3,4-j][1,7]benzodiazonin-1-one, staurosporine derivatives such as disclosed in EP0296110, e.g. midostaurin; 2,3,10,11,12,13-hexahydro-10-methoxy-9-methyl-11-(methylamino)-, (9S,10R,11R,13R)-(9CI); tyrphostin 51; and hypericin, which is also known as phenanthro[1,10,9,8-opqra]perylene-7,14-dione, 1,3,4,6,8,13-hexahydroxy-10,11-dimethyl-, stereoisomer (6CI,7CI,8CI,9CI), UCN-01,safingol, BAY 43-9006, bryostatin 1, perifosine; llmofosine; RO 318220 and RO 320432; GO 6976; Isis 3521; LY333531/LY379196. The term “a PKC delta kinase inhibitor”, as used herein, relates to a compound which targets, decreases or inhibits the delta isozymes of PKC. The delta isozyme is a conventional PKC isozymes and is Ca²⁺-dependent. An example of a PKC delta kinase inhibitor includes, but is not limited to, Rottlerin, which is also known as 2-Propen-1-one, 1-[6-[(3-acetyl-2,4,6-trihydroxy-5-methylphenyl)methyl]-5,7-dihydroxy-2,2-dimethyl-2H-1-benzopyran-8-yl]-3-phenyl-, (2E)-(9CI).
liii. a polyamine synthesis inhibitor; which targets, decreases or inhibits polyamines spermidine; such as DMFO, which is also known as (−)-2-difluoromethylornithin; N1, N12-diethylspermine 4HCl. The polyamines spermidine and spermine are of vital importance for cell proliferation, although their precise mechanism of action is unclear. Tumor cells have an altered polyamine homeostasis reflected by increased activity of biosynthetic enzymes and elevated polyamine pools.
liv. a proteosome inhibitor; which targets, decreases or inhibits proteasome, such as aclacinomycin A; gliotoxin; PS-341; MLN 341; bortezomib; velcade. Examples of targets of a proteosome inhibitor include, but are not limited to, O(2)(−)-generating NADPH oxidase, NF-kappaB, and/or farnesyltransferase, geranyltransferase I.
lv. a PTP1B inhibitor; which targets, decreases or inhibits PTP1B, a protein tyrosine kinase inhibitor; such as L-leucinamide, N-[4-(2-carboxyethenyl)benzoyl]glycyl-L-α-glutamyl-,(E).
lvi. a protein tyrosine kinase inhibitor including a SRC family tyrosine kinase inhibitor; a Syk tyrosine kinase inhibitor; and a JAK-2 and/or JAK-3 tyrosine kinase inhibitor; The term “a protein tyrosine kinase inhibitor”, as used herein, relates to a compound which targets, decreases or inhibits protein tyrosine kinases. Protein tyrosine kinases (PTKs) play a key role in the regulation of cell proliferation, differentiation, metabolism, migration, and survival. They are classified as receptor PTKs and non-receptor PTKs. Receptor PTKs contain a single polypeptide chain with a transmembrane segment. The extracellular end of this segment contains a high affinity ligand-binding domain, while the cytoplasmic end comprises the catalytic core and the regulatory sequences. Examples of targets of a tyrosine kinase inhibitor include, but are not limited to, ERK1, ERK2, Bruton's tyrosine kinase (Btk), JAK2, ERK ½, PDGFR, and/or FLT3. Examples of indirect targets include, but are not limited to, TNFalpha, NO, PGE2, IRAK, iNOS, ICAM-1, and/or E-selectin. Examples of a tyrosine kinase inhibitor include, but are not limited to, tyrphostin AG 126; tyrphostin Ag 1288; tyrphostin Ag 1295; geldanamycin; and genistein.
Non-receptor tyrosine kinases include members of the Src, Tec, JAK, Fes, Abl, FAK, Csk, and Syk families. They are located in the cytoplasm as well as in the nucleus. They exhibit distinct kinase regulation, substrate phosphorylation, and function. Deregulation of these kinases has also been linked to several human diseases. The term “a SRC family tyrosine kinase inhibitor”, as used herein, relates to a compound which targets, decreases or inhibits SRC. Examples of a SRC family tyrosine kinase inhibitor include, but are not limited to, PP1, which is also known as 1H-pyrazolo[3,4-d]pyrimidin-4-amine, 1-(1,1-dimethylethyl)-3-(1-naphthalenyl)-(9CI); and PP2, which is also known as 1H-Pyrazolo[3,4-d]pyrimidin-4-amine, 3-(4-chlorophenyl)-1-(1,1-dimethylethyl)-(9CI).
The term “a Syk tyrosine kinase inhibitor”, as used herein, relates to a compound which targets, decreases or inhibits Syk. Examples of targets for a Syk tyrosine kinase inhibitor include, but are not limited to, Syk, STAT3, and/or STAT5. An example of a Syk tyrosine kinase inhibitor includes, but is not limited to, piceatannol, which is also known as 1,2-benzenediol, 4-[(1E)-2-(3,5-dihydroxyphenyl)ethenyl]-(9CI).
The term “a Janus (JAK-2 and/or JAK-3) tyrosine kinase inhibitor”, as used herein, relates to a compound which targets, decreases or inhibits janus tyrosine kinase. Janus tyrosine kinase inhibitor are shown anti-leukemic agents with anti-thrombotic, anti-allergic and immunosuppressive properties. Targets of a JAK-2 and/or JAK-3 tyrosine kinase inhibitor include, but are not limited to, JAK2, JAK3, STAT3. An indirect target of an JAK-2 and/or JAK-3 tyrosine kinase inhibitor includes, but is not limited to CDK2. Examples of a JAK-2 and/or JAK-3 tyrosine kinase inhibitor include, but are not limited to, Tyrphostin AG 490; and 2-naphthyl vinyl ketone.
Compounds which target, decrease or inhibit the activity of c-Abl family members and their gene fusion products, e.g. include PD180970; AG957; or NSC 680410.
lvii. a retinoid; which target, decrease or inhibit retinoid dependent receptors; such as isotretinoin, tretinoin, alitretinoin, bexarotene.
lviii. a RNA polymerase II elongation inhibitor; which targets, decreases or inhibits insulin-stimulated nuclear and cytosolic p70S6 kinase in CHO cells; targets, decreases or inhibits RNA polymerase II transcription, which may be dependent on casein kinase II; and targets, decreases or inhibits germinal vesicle breakdown in bovine oocytes; such as 5,6-dichloro-1-beta-D-ribofuranosylbenzimidazole.
lvix. a serine/threonine kinase inhibitor; which inhibits serine/threonine kinases; such as 2-aminopurine. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-kappaB, elF2alpha, COX2, RANTES, IL8, CYP2A5, IGF-1, CYP2B1, CYP2B2, CYP2H1, ALAS-1, HIF-1, erythropoietin, and/or CYP1A1.
lx. a sterol biosynthesis inhibitor; which inhibits the biosynthesis of sterols such as cholesterol; such as terbinadine. Examples of targets for a sterol biosynthesis inhibitor include, but are not limited to, squalene epoxidase, and CYP2D6.
lxi. a topoisomerase inhibitor; including a topoisomerase I inhibitor and a topoisomerase II inhibitor. Examples of a topoisomerase I inhibitor include, but are not limited to, topotecan, gimatecan, irinotecan, camptothecan and its analogues, 9-nitrocamptothecin and the macromolecular camptothecin conjugate PNU-166148 (compound A1 in WO9917804); 10-hydroxycamptothecin e.g. the acetate salt; idarubicin, e.g. the hydrochloride; irinotecan, e.g. the hydrochloride; etoposide; teniposide; topotecan, topotecan hydrochloride; doxorubicin; epirubicin, epirubicin hydrochloride; mitoxantrone, mitoxantrone, e.g. the hydrochloride; daunorubicin, daunorubicin hydrochloride, vairubicin, dasatinib (BMS-354825). Irinotecan can be administered, e.g., in the form as it is marketed, e.g., under the trademark CAMPTOSAR®. Topotecan can be administered, e.g., in the form as it is marketed, e.g., under the trademark HYCAMTIN®. The term “topoisomerase II inhibitor”, as used herein, includes, but is not limited to, the anthracyclines, such as doxorubicin, including liposomal formulation, e.g., CAELYX®, daunorubicin, including liposomal formulation, e.g., DAUNOSOME®, epirubicin, idarubicin and nemorubicin; the anthraquinones mitoxantrone and losoxantrone; and the podophillotoxines etoposide and teniposide. Etoposide is marketed as ETOPOPHOS®; teniposide as VM 26-BRISTOL®; doxorubicin as ADRIBLASTIN® or ADRIAMYCIN®; epirubicin as FARMORUBICIN® idarubicin as ZAVEDOS®; and mitoxantrone as NOVANTRON®.
lxii. VEGFR tyrosine kinase inhibitor; which targets, decreases and/or inhibits the known angiogenic growth factors and cytokines implicated in the modulation of normal and pathological angiogenesis. The VEGF family (VEGF-A, VEGF-B, VEGF-C, VEGF-D) and their corresponding receptor tyrosine kinases [VEGFR-1 (Flt-1), VEGFR-2 (Flk-1, KDR), and VEGFR-3 (Flt-4)] play a paramount and indispensable role in regulating the multiple facets of the angiogenic and lymphangiogenic processes. An example of a VEGFR tyrosine kinase inhibitor includes 3-(4-dimethylaminobenzylidenyl)-2-indolinone. Compounds which target, decrease or inhibit the activity of VEGFR are especially compounds, proteins or antibodies which inhibit the VEGF receptor tyrosine kinase, inhibit a VEGF receptor or bind to VEGF, and are in particular those compounds, proteins or monoclonal antibodies generically and specifically disclosed in WO9835958, e.g. 1-(4-chloroanilino)-4-(4-pyridylmethyl) phthalazine or a pharmaceutical acceptable salt thereof, e.g. the succinate, or in WO0009495, WO0027820, WO0059509, WO9811223, WO0027819 and EP0769947; e.g. those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO0037502 and WO9410202; Angiostatin, described by M. S. O'Reilly et al, Cell 79, 1994, 315-328; Endostatin described by M. S. O'Reilly et al, Cell 88, 1997, 277-285; anthranilic acid amides; ZD4190; ZD6474 (vandetanib); SU5416; SU6668; or anti-VEGF antibodies or anti-VEGF receptor antibodies, e.g. RhuMab (bevacizumab). By antibody is meant intact monoclonal antibodies, polyclonal antibodies, multispecific antibodies formed from at least 2 intact antibodies, and antibodies fragments so long as they exhibit the desired biological activity. an example of an VEGF-R2 inhibitor e.g. includes axitinib,
lxiii. a gonadorelin agonist, such as abarelix, goserelin, goserelin acetate,
lxiv. a compound which induce cell differentiation processes, such as retinoic acid, alpha-, gamma- or 8-tocopherol or alpha-, gamma- or 8-tocotrienol.
lxv. a bisphosphonate, e.g. including etridonic, clodronic, tiludronic, pamidronic, alendronic, ibandronic, risedronic and zoledronic acid.
lxvi. a heparanase inhibitor which prevents heparan sulphate degradation, e.g. PI-88,
lxvii. a biological response modifier, preferably alymphokine or interferons, e.g. interferon alpha,
lxviii. a telomerase inhibitor, e.g. telomestatin,
lxix. mediators, such as inhibitors of catechol-O-methyltransferase, e.g. entacapone,
lxx: ispinesib, sunitinib (SU11248), diethylstilbestrol (DES), BMS224818 (LEA29Y),
lxxi somatostatin or a somatostatin analogue, such as octreotide (Sandostatin® or Sandostatin LAR®).
lxxii. Growth Hormone-Receptor Antagonists, such as pegvisomant, filgrastim or pegfilgrastim, or interferon alpha:
lxxiii. monoclonal antibodies, e.g. useful for leukemia (AML) treatment, such as alemtuzumab (Campath®), rituximab/Rituxan®), gemtuzumab, (ozogamicin, Mylotarg®), epratuzumab.
lxxiv. altretamine, amsacrine, asparaginase (Elspar®), denileukin diftitox, masoprocol, pegaspargase.
lxxv. a phosphodiesterase inhibitor, e.g. anagrelide (Agrylin®, Xagrid®).
lxxvi. a cancer vaccine, such as MDX-1379.
Ixxvii. a methylmalonic acid lowering agent (see e.g. WO2002002093).

Cancer treatment with a combination of the present invention, optionally in combination with another drug substance, may be associated with radiotherapy. Cancer treatment with a combination of the present invention, optionally in combination with another drug substance, may be a second line treatment, e.g. following treatment with another anticancer drug or other cancer therapy.

Anesthetics which are prone to be useful as a combination partner, e.g. in combination therapy, with a combination of the present invention e.g. include ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocalne, and phenazopyridine.

Antidiarrheal drug substances which are prone to be useful as a combination partner, e.g. in combination therapy, with a combination of the present invention, e.g. include diphenoxylate, loperamide, codeine.

For example a combination according to the present invention may be used in combination with a methylmalonic acid lowering agent (see e.g. WO2002002093). For example a combination according to the present invention may be used in combination with a platinum compound, e.g. cisplatin.

Other drug substances may be used in free form, in salt form, e.g. pharmaceutically acceptable salt form, optionally in the form of a solvate, optionally in the form of a pharmaceutically acceptable pro-drug, e.g. including carboxylic acid derivatives of carboxylic acid containing drug substances, such as a pharmaceutically acceptable carboxylic acid esters. Such other drug substance may be in the form of pure isomers or in any isomeric mixture where isomers may exist.

If a combination of the present invention is administered in combination with another drug substance dosages of the co-administered other drug substance will of course vary depending on the type of co-drug employed, on the specific drug employed, on the condition being treated. In general dosages similar than those as provided by the supplier of the other drug substance may be appropriate. Appropriate dosing of another drug substance may be performed as appropriate, e.g. according, e.g. analogously, as indicated for a specific drug in literature or internet.

As used herein, the term “toxicity” refers to a toxic event associated with the administration on an antifolate, see e.g. “Antifolate Drugs in Cancer Therapy”, Humana Press, see e.g. “Book review—Antifolate Drugs in Cancer Therapy by Ann L. Jackman. Author: Nair M. G. 1. Source: Drug Discovery Today, Volume 4, Number 11, 1999”.

### Methods

To assess the effect of an mTOR inhibitor on the antitumor efficacy of a compound of in a human tumor xenograft model, female nude mice bearing human MX-1 breast carcinoma may be treated with an antifolate compound, e.g. a compound of formula I U.S. Pat. No. 5,344,932 alone, or in combination with an mTOR inhibitor.

The animals may be maintained on sterilized standard lab chow ad libitum and sterilized water ad libitum. The human MX-1 tumor cells (5*10<6>) obtained from donor tumors may be implanted subcutaneously in a thigh of female nude mice 8- to 10-weeks old. Beginning on day 7 post tumor cell implantation, the animals may be treated with a compound of formula I U.S. Pat. No. 5,344,932 (100 mg/kg or 150 mg/kg) once daily on days 7 through 11 and 14 through 18 by intraperitoneal injection alone or along with an mTOR inhibitor (oral treatment, 1 to 30 mg/kg).

Tumor response may be monitored by tumor volume measurements twice weekly over the course of the experiment. Toxicity may be monitored by body weight measurements made at the same time as the tumor volume measurements. Tumor growth delay is meant to be the difference in days for the treated and the controls tumors to reach 1000 mm.

The human MX-1 breast carcinoma xenograft is responsive to treatment with a compound of formula I U.S. Pat. No. 5,344,932 with doses of 100 mg/kg and 150 mg/kg producing tumor growth delays of 17 days and 21 days, respectively. An mTOR inhibitor administered to the animals alone at two doses 5 mg/kg and 20 mg/kg on the same schedule as a compound of formula I U.S. Pat. No. 5,344,932 may produce tumor growth delays of several days.

Combinations of a compound of formula I U.S. Pat. No. 5,344,932 may be administered along with an mTOR inhibitor as simultaneous combination regimens, or an mTOR inhibitor may be administered before administration of a compound of formula I U.S. Pat. No. 5,344,932, or a compound of formula I U.S. Pat. No. 5,344,932, may be administered before administration of an mTOR inhibitor. Administration of a compound of formula I U.S. Pat. No. 5,344,932, in combination with an mTOR inhibitor may reduce the delay of tumor growth.

Body weight may be used as a general measure of toxicity for each of the treatment regimens. The body weight loss pattern may reflect the treatment regimens with weight decrease during the treatment times of days 7 through 11 and 14 through 18 with some weight recovery during the intervening two days. The weight loss due to a compound of formula I U.S. Pat. No. 5,344,932, may be dose dependent but overall minor. The animals treated with a compound of formula I U.S. Pat. No. 5,344,932 and an mTOR inhibitor may gain weight over the course of the experiment.

The timing of administering a compound of formula I U.S. Pat. No. 5,344,932 and an mTOR inhibitor may be varied.

### Clinical Trials

Investigated is an open label, multicenter Phase I study investigating the combination of Compound A with the standard treatment regimen of permetrexed in patients with advanced (unresectable or metastatic) NSCLC previously treated with one line of chemotherapy. A maximum of 120 patients are enrolled.

### Study Objectives

To confirm the feasible dose levels/regimens of RAD001 combined with the standard permetrexed regimen in patients with NSCLC previously treated with one regimen of chemotherapy based on the evaluation of safety and ability to deliver the required dose intensity of permetrexed

To confirm the PK of RAD001 in NSCLC patients treated with the combination RAD001 and permetrexed and to estimate the PK interaction between RAD001 and permetrexed.

To confirm the clinical efficacy of combined administration of different RAD001 dose levels/regimens with standard permetrexed treatment based on the evaluation of overall tumour response according to RECIST.

The study is designed as a Bayesian sequential dose-escalation scheme based on:
a time-to-event model of the occurrence of DLTs estimating the probability that patients experience a DLT within their first cycle of treatment (“End-of-Cycle-1 DLT rate”)

The study is designed as a Bayesian sequential dose-escalation scheme based on
a time-to-event model of the occurrence of DLTs estimating the probability that patients experience a DLT within their first cycle of treatment (“End-of-Cycle-1 DLT rate”)
   and
a model estimating the probability that the Relative Dose Intensity (RDI) of permetrexed is optimal versus sub-optimal.

Successive dose levels are investigated initially in 2 separate treatment arms, each corresponding to a different dosing schedule for Compound A:
Arm 1: continuous daily dosing of Compound A in combination with the standard 21-day treatment cycle of permetrexed
Arm 2: continuous weekly dosing of Compound A (Days 1, 8, 15) in combination with the standard 21-day treatment cycle of permetrexed

After the completion of the evaluation of continuous daily regimen of Compound A in arm 1 a further Compound A regimen may be initiated as follows:
Interrupted daily dosing of Compound A with no administration of Compound A between Days 15 and 21 of each cycle of therapy)

Patients are randomized and centrally allocated to either arm and are enrolled at the current dose level/regimen. Dose escalation can occur in parallel, although independently, in each arm.

RDI is evaluated when the first 15 patients in each arm have been followed for at least 2 cycles and this time point will be considered an interim analysis look. If the RDI evaluation contradicts the dose-escalation decision based on the safety signals, then a second interim look during the study is to be planned. The RDI is evaluated as well at the end of the trial.

The study investigates two different dosing schedules of Compound A (daily and weekly) in combination with the standard 21-day cyclic administration of permetrexed. Within each Compound A dosing schedule, two different regimens, continuous and interrupted dosing, are investigated and up to three individual dose levels of RAD001 per regimen are explored

The Compound A (C-A) doses within the two arms are as follows:

RANDOMIZATION

| | ARM 2 WEEKLY |
|---|---|
| ARM 1 DAILY C-A DOSING^{a} | C-A DOSING^{a} |
| | 1^{ST} REGIMEN - WEEKLY |
| 1^{ST} REGIMEN - DAILY | CONTINUOUS (QW) |
| CONTINUOUS (QD) | C-A Dosing Days 1, 8 |
| C-A Dosing Days 1-21 of each cycle^{b} | and 15 of each cycle^{b} |

| | | | |
|---|---|---|---|
| QD5^{c} | C-A 5 mg/day | QW30^{c} | C-A 30 mg/week |
| QD10 | C-A 10 mg/day | QW50 | C-A 50 mg/week |
| QD2.5 | RAD001 2.5 mg/day | QW20 | C-A 20 mg/week |

| | |
|---|---|
| 2^{ND} REGIMEN - DAILY | 2^{ND} REGIMEN - WEEKLY |
| INTERRUPTED (ID) | INTERRUPTED(QW) |
| RAD001 Dosing Days | RAD001 Dosing Days 1 and 8 |
| 1-14 of each cycle^{b} | of each cycle^{b} |

| | | | |
|---|---|---|---|
| ID5 | C-A 5 mg/day | QW30 | C-A 30 mg/week |
| ID10 | C-A 10 mg/day | QW50 | C-A 50 mg/week |
| ID2.5 | C-A 2.5 mg/day | QW20 | C-A 20 mg/week |

^{a}All patients receive C-A in combination with permetrexed chemotherapy aiming at 6 cycles of treatment
^{b}In Cycle 1, C-A dosing beginy on Day 2
^{c}Starting dose level

Compound A dosing is commenced on Day 2 of Cycle 1 in both arms, just before permetrexed infusion. The decision to escalate the dose of Compound A will depend on the estimated end-of-cycle 1 DLT rate and on the RDI of the chemotherapy partner of Compound A. Patients will continue to receive Compound A until progression of disease or unacceptable toxicity.

### Compound A

### Permetrexed Doses

All patients receive their respective Compound A doses alongside the standard 21-day cycle of permetrexed, applying standard pre-medication, dose interruption and adjustment (see appendix).
Permetrexed is administered on Day 1 at a dose of 500 mg/m² as a 10 min. intravenous infusion.

Patients may receive up to a maximum of 6 cycles of permetrexed.

Endpoints: Dose limiting toxicities occurring at any time, relative dose intensity of chemotherapy.

The study is aimed at providing the patients six cycles of permetrexed chemotherapy. One cycle is defined as an interval between two consecutive administrations of chemotherapy regimens (with a default of 21-days) and Day 1 for each cycle is defined as the day of the initiation of the chemotherapy. During each cycle, patients have regular safety laboratory tests on a weekly basis (Days 8 and 15). On Day 1 they will also undergo a full safety assessment before further chemotherapy is initiated.

Only in Cycle treatment with RAD001 is delayed until Day 2 to allow serial blood sampling for the evaluation of baseline PK profiles of permetrexed on Day 1. The PK blood sampling is continued on Day 8 of Cycle 1 (baseline PK profile of RAD001), and on Day 1 of Cycle 2 (combination PK profiles for both study drugs are administered together).

Computerized tomography (CT) scans for tumor measurements is performed routinely every six weeks (after every 2 cycles of permetrexed) while the patient is receiving chemotherapy. Following completion or discontinuation of chemotherapy, all patients may continue to receive RAD001 on a continuous daily or weekly basis (irrespective of the RAD001 regimen while on chemotherapy) until progressive disease or unacceptable toxicity occurs. In such cases, CT scans are carried out every 6 weeks.

Efficacy is evaluated using the following endpoints: Overall response rate (ORR) according to RECIST (optionally amended RESIST), early progression rate (EPR) according to RECIST (optionally amended RESIST). Tumor assessment is performed by a CT scan throughout the study Drug-drug interaction is evaluated from the comparison of study levels when administered alone or in combination.

In the clinical trials COMPOUND A in combination with permetrexed show appropriate efficacy regarding tumor growth. The combination of COMPOUND A with permetrexed shows synergistic effects in several aspects.

1. A combination comprising an mTOR inhibitor and an antifolate compound.

2. A pharmaceutical composition comprising an mTOR inhibitor and an antifolate compound in combination beside pharmaceutically acceptable excipient.

3. A pharmaceutical package comprising an mTOR inhibitor, an antifolate compound, and instructions for combined administration.

4. A pharmaceutical package comprising an mTOR inhibitor, and instructions for combined administration with an antifolate compound.

5. A pharmaceutical package comprising an antifolate compound and instructions for combined administration with an mTOR inhibitor.

6. A method for the treatment of disorders which are mediated by mTOR and/or thymidylate synthetase, comprising administering to a subject in need thereof a therapeutically effective amount of an mTOR inhibitor in combination with an antifolate compound, either in sequence or simultaneously.

7. (canceled)

8. A method for the preparation of a medicament for use in a method according to claim 6, comprising either
a) combining, e.g. mixing, an mTOR inhibitor and an antifolate compound with pharmaceutically acceptable excipient, or
b) combining e.g. mixing, an mTOR inhibitor with pharmaceutically acceptable excipient to obtain a pharmaceutical composition COMP1, and combining, e.g. mixing, an antifolate compound, with pharmaceutically acceptable excipient to obtain a pharmaceutical composition COMP2, and either
(i) combining the pharmaceutical composition COMP1 and the pharmaceutical composition COMP2 in one single package, or
(ii) packing the pharmaceutical composition COMP1 separately and packing the pharmaceutical composition COMP2 separately, but adding to each package instructions for combined administration of pharmaceutical composition COMP1 and pharmaceutical composition COMP2.

9. (canceled)

10. A combination according to claim 1, wherein said mTOR inhibitor is rapamycin, 40-O-(2-hydroxyethyl)-rapamycin, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-0-(2-hydroxyethyl)-rapamycin, 40-[3-hydroxy-2-(hydroxy-methyl)-2-methylpropanoate]-rapamycin or biolimus.

11. A combination according to claim 10, wherein said mTOR inhibitor is 40-O-(2-hydroxyethyl)-rapamycin.

12. A combination according to claim 1, wherein said antifolate compound is a compound of formula 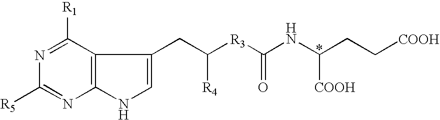
wherein
R₁ is —OH or —NH₂;
R₃ is 1,4-phenylene or 1,3-phenylene unsubstituted or substituted with chloro, fluoro, methyl, methoxy, or trifluoromethyl; thienediyl or furanediyl unsubstituted or substituted with chloro, fluoro, methyl, methoxy, or trifluoromethyl; cyclohexanediyl; or alkanediyl;
R₄ is hydrogen, methyl, or hydroxymethyl; and
R₅ is hydrogen, alkyl of 1 to 6 carbon atoms, or amino;
in free form or in the form of a pharmaceutically acceptable salt; optionally in the form of a solvate.

13. A combination according to claim 12 wherein the antifolate compound is permetrexed of formula

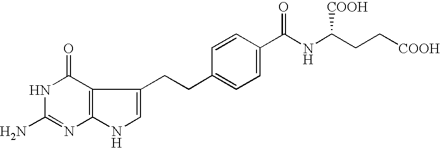

14. A combination according to claim 13, wherein permetrexed is in the form of a disodium salt in the form of a heptahydrate.
